(19) United States
(12) Patent Application Publication
(10) Pub. No.: US 2021/0383667 A1
(43) Pub. Date: Dec. 9, 2021

(54) METHOD FOR COMPUTER VISION-BASED ASSESSMENT OF ACTIVITIES OF DAILY LIVING VIA CLOTHING AND EFFECTS

(71) Applicant: KONINKLIJKE PHILIPS N.V., EINDHOVEN (NL)

(72) Inventors: Daniel Jason SCHULMAN, Jamaica Plain, MA (US); Christine Menking SWISHER, San Diego, CA (US)

(21) Appl. No.: 17/285,795

(22) PCT Filed: Oct. 15, 2019
(86) PCT No.: PCT/EP2019/077862

§ 371 Date: Apr. 15, 2021

Related U.S. Application Data
(60) Provisional application No. 62/746,190, filed on Oct. 16, 2018.

(51) Int. Cl. G08B 21/04; G06T 7/11; G06K 9/62; G06N 3/04; G06K 9/46; G06K 9/32; A61B 5/00

(52) U.S. Cl. CPC G08B 21/0423; G06T 7/11; G06K 9/6256; G06N 3/04; G06K 9/4609; G06T 2207/10004; A61B 5/0022; G08B 21/0415; G06T 2207/20081; G06T 2207/10016; G06K 9/3233

(57) ABSTRACT

A method of detecting decline in activities of daily living (ADLs) over time, the method including gathering a plurality of image data of a subject over a period of time, preprocessing the image data to obtain a plurality of standardized images, segmenting out a feature from each of the image data, providing the segmented features to a trained model to identify possible changes in the features over time, classifying the possible changes as evidence, and using the evidence to calculate a risk score.

### TECHNICAL FIELD

Embodiments described herein relate generally to assessments of performance of activities of daily living (ADLs), and in particular to detecting deterioration of seniors aging-in-place and others at risk of cognitive and/or physical decline.

### BACKGROUND

For seniors aging-in-place and others at risk of hospitalization, loss of independence, or other adverse outcomes, performance of activities of daily living (ADLs) can be an indicator of decline in functional health status or unmet health needs. Several ADLs including dressing oneself and performing personal hygiene have been characterized as “early-loss” ADLs. Deficiencies in these ADLs may appear early in a process of functional decline, especially in decline of cognitive functioning toward dementia.

Standardized assessments of ADL performance, such as checklists or questionnaires, are available and in broad use, relying variously on self-reporting by a senior, or on observation by a provider or a formal or informal caregiver. Self-reporting assessments by the senior may place a high burden on the senior, especially for seniors with cognitive impairments who may have difficulty with recall, and self-reporting assessments may be subject to bias. For example, seniors may avoid reporting socially undesirable deficiencies such as a difficulty in performing personal hygiene.

Some sensors (e.g., wearables) require the senior to wear, charge, or otherwise take action. Seniors may forget or choose not to wear or use the sensor. This can cause automated sensor-based assessment to suffer some of the same problems as self-reporting assessment. Seniors with cognitive impairment are more likely to forget, for example, to wear a device, and seniors may avoid wearing a device seen as socially undesirable, either because of the appearance or form of the device itself or because of concerns, as above, of others learning of embarrassing deficiencies.

Assessment by trained professionals has high cost (e.g., to account for training of assessors) and requires extensive, obtrusive monitoring of the senior. For seniors with relatively little impairment (e.g., the “early loss” group referenced above) who are aging-in-place with little daily assistance, ongoing and repeated assessment by trained professionals would require ongoing visible intrusion into daily life. Alternatively, allowing longer intervals between assessments (e.g., a yearly assessment) increases the risk of missing more abrupt changes in health and functional status.

### SUMMARY

A brief summary of various embodiments is presented below. Some simplifications and omissions may be made in the following summary, which is intended to highlight and introduce some aspects of the various embodiments, but not to limit the scope of the invention. Detailed descriptions of embodiments adequate to allow those of ordinary skill in the art to make and use the inventive concepts will follow in later sections.

Embodiments include a method of detecting decline in activities of daily living (ADLs) over time, the method including gathering a plurality of image data of a subject over a period of time, preprocessing the image data to obtain a plurality of standardized images, segmenting out a feature from each of the image data, providing the segmented features to a trained model to identify possible changes in the features over time, classifying the possible changes as evidence, and using the evidence to calculate a risk score.

The image data may be still image data. The image data may be video image data. The feature may be an article of clothing or a bodily feature.

The trained model may be a convolutional neural network (CNN). The CNN may detect the possible changes as no change over a threshold period as evidence of declining ADL capabilities.

The risk score may be reported to a health care management entity.

The method may include detecting a lack of personal hygiene and repeated use of clothing based on the segmented features, and determining that the lack of personal hygiene and repeated use of clothing are evidence of an ADL deficiency. The detecting may include capturing images of a same clothing item over at least three days.

Embodiments may also include a detection system including a plurality of image sources to obtain a plurality of images of a subject at periodic intervals, at least one image preprocessing module configured to preprocess the plurality of images to obtain standardized images, a clothing and effects localization/segmentation component configured to apply techniques to the plurality of images to separate parts of the plurality of images [clothing and personal effects] via segmentation and/or localization, and an activity of daily living (ADL) evidence classification module configured to translate the information into evidence for or against ADL deficiencies.

The images may be from still or video feeds. The image sources may include one of telehealth and check-in video, social media, or in-home devices. The image sources may provide images at scheduled time intervals.

The detection system may be configured to produce images with a greater than ninety percent probability, or other specified probability, of being the subject at an appropriate time and place

Outputs from the clothing and effects localization/segmentation component may include images with associated masks to indicate which pixels of the image are clothing and personal effects and/or bounding boxes around a region of interest.

In the clothing and effects segmentation/localization module, preprocessed images may be identified and classified into different groups for comparison with stored images.

Images may be classified into clothing groups of the subject, facial and body images of the subject, embarrassing or unusable images of the subject, images that are not the subject, and images of blank space that do not include the subject.

The ADL evidence classification module may include a temporal comparison module which examines similarity of different articles of clothing to determine whether two or more time related clothing items are the same.

The ADL evidence classification module may be configured to produce raw scores of whether clothes are dirty or disheveled.

The detection system may include a risk detection component configured to identify a risk whenever cumulative ADL deficiency evidence is above a specified threshold within a specified time period.

The detection system may include a risk detection module configured to detect when ADL evidence indicates the presence of ADL deficiency with increased risk of adverse events.

The detection system may include a risk detection module to produce a structured risk report when cumulative ADL deficiency evidence is above a specified threshold, the structured risk report describing the ADL deficiency and a resultant risk. The risk report may be annotated with images of ADL evidence that was detected.

### BRIEF DESCRIPTION OF THE DRAWINGS

Additional objects and features of the invention will be more readily apparent from the following detailed description and appended claims when taken in conjunction with the drawings. Although several embodiments are illustrated and described, like reference numerals identify like parts in each of the figures, in which:

FIG. 1 illustrates a system overview different stages of monitoring, processing, and reporting deficiencies in ADLs in accordance with embodiments described herein; and

FIG. 2 illustrates a multi-task convolutional neural network (CNN) configured to perform face detection and clothing segmentation in accordance with FIG. 1.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application.

For subjects categorized as seniors or at risk of cognitive and/or physical decline for other reasons, two activity groups may be established: ADL and instrumental ADL (IADL). Subjects with IADL deficiencies may engage in activities that include higher functioning, more complex tasks. Subjects with IADL deficiencies having few or no deficits can live independently with infrequent assistance, performing tasks such as grocery shopping. Subjects with IADL deficiencies can be relatively independent with an assistance home health aide stopping by infrequently.

A subject with deficits in the performance of ADLs has more limitations and restrictions. Embodiments described herein involve people at risk for the development of ADL deficiencies, with or without substantial deficiencies in the performance of IADLs. Individuals in this category may have some cognitive impairment, but embodiments are not limited thereto.

Embodiments described herein are concerned with individuals who are aging in-place and/or community-dwelling. Aging in place can refer to seniors living in their own homes, and community-dwelling similarly can refer to individuals living in their own home. Some seniors and other individuals may be at a risk of loss of independence or activities. Methods are discussed for ongoing assessment of activities in daily living performance, such as dressing and personal hygiene. Changes in those parameters may be indicators of a variety of problems including cognitive impairment, among others. Embodiments describe using image-based analysis of clothing and personal appearance to classify whether a subject is actively engaged in these activities in a successful manner

Embodiments may avoid pervasive and continuous video monitoring. Such monitoring may not be favored by consumers. Continuous monitoring includes checking on what a person is doing at arbitrary times, with the goal of capturing activity at a specific time, such as whether someone is dressing themselves in the morning. Adding other activities may be technically difficult such as installing cameras in many locations in the home, which has high cost and low acceptance.

Consumers have higher acceptance of daily video check-ins, or daily phone check-ins. Embodiments described herein use technology to augment these activities.

Automatic sensing and detection of ADL performance is difficult, and reaching acceptable accuracy is a topic of ongoing research. Some work focuses on detecting when an ADL is performed, without additionally assessing whether performance of the ADL was successful (e.g., did the subject successfully dress him/herself) or the amount of difficulty or effort required, both of which are useful in assessment of a deficiency in ability of perform ADLs. Also, individuals may develop coping habits which can mask an underlying deficiency, such as wearing the same clothes several days in a row to cope with difficulty in dressing and/or personal hygiene.

Monitoring and assessment of ADLs, especially early-loss ADLs, has value to multiple stakeholders including formal and informal (e.g., friends and family) caregivers, health care providers, and health care organizations, with applications ranging from risk prediction for targeting of interventions to supporting peace of mind for remote family caregivers.

Automatic ADL detection and assessment may use a variety of sensing technologies including wearable accelerometers and accelerometer-equipped devices (e.g., smartphones, fitness watches), and unobtrusive sensing methods including cameras and computer vision, acoustic sensing, and radar (e.g., WiFi). Methods and devices such as these may detect when an ADL is being performed and, in some cases, whether ADL performance is successful. Summarizing ADL performance over a sufficient span of time may provide an assessment of ADL deficiency. Summarizing trends or changes in ADL performance over a sufficient span of time may provide an assessment of ADL decline.

The performance (or lack of performance, or unsuccessful performance) of some ADLs may leave evidence that can be observed later. In the case of dressing and personal hygiene ADLs, evidence for the performance of the ADLs may be observed in the state of a client's clothing, grooming (e.g., hair), and personal effects. The change in these items may be observed over time (e.g., whether the same clothes are worn over multiple days).

A set of computer vision methods may be applied to perform assessment of dressing and personal hygiene ADLs. These methods supply reliable components to identify clothing and personal effects in an image, and to classify a pair of images as having the same clothing or different clothing. Using these components, embodiments described herein implement an ADL assessment that, based on images of a subject such as a senior, provide an automated judgement of whether the images include evidence that dressing and personal hygiene ADLs have been performed successfully or unsuccessfully.

According to embodiments described herein, a detection and reporting system may tell if someone has been dressing themselves or performing personal hygiene by using machine vision to view their clothes and/or personal appearance using one or various camera angles over the course of several days. Machine vision can detect small changes in appearance that may not be apparent to the naked eye of an untrained human observer and does not require the consistent participation of a single observer. If someone is wearing disheveled clothing, or if someone is wearing the same clothing for several days, small changes may be detected and classified by the system. Image capturing may be performed by taking still images or by using small snippets of video. These images may be accurately analyzed for change, even if analyzed only once or twice per day.

Personal hygiene may include analysis or hair style or length. If a person normally prepares their hair in a certain way, the system may store data about a subject and determine small changes thereto that could be an indication of mental impairment. Likewise, a subject may have a shaving routine that results in facial hair appearing a certain way. When this routine deviates, the system may be able to pick up fine changes that a person could not detect.

Personal hygiene markers may include a condition of a subject's hair, the length of it, the color of it, or the cleanliness of it. Personal hygiene may also include the cleanliness of a person's face. The system may determine whether a subject's face is dirty, or if facial hair had not been appropriately trimmed

A subject's clothing may be inspected for irregularities. In one example, if a person wears the same clothes such as the same pants or shirt for consecutive days, or for a predetermined amount of days, such as three, the system can be programmed to detect and report the occurrence. Similarly, a subject may look disheveled, such as a subject's shirt being untucked, or a button-down shirt improperly buttoned. Front, back, or side images may reveal that a shirt tail is tucked in one place and untucked in another. Images may be scanned to reveal that buttons have been broken off and are missing. Images or videos may reveal that clothes are dirty and have remained so for multiple days.

Images or videos may reveal that a subject is not wearing their glasses for an extended period. The system may provide an alert such that a caregiver could intervene and look for the eyeglasses in a vicinity of the subject. Images or videos could reveal a bruise on the body of a subject, such as if the subject fell, bumped into an object, or dropped something upon themselves.

In addition to hygiene, personal appearance, and clothing aberrations, other changes to a subject's living condition could be reflected in changes or lack of changes to the subject's home environment. If chairs are normally aligned in a manner, or a subject's bed is normally made in certain manner, slight changes to these configurations could be detected by the system and reported to a higher authority when the changes exceed a threshold.

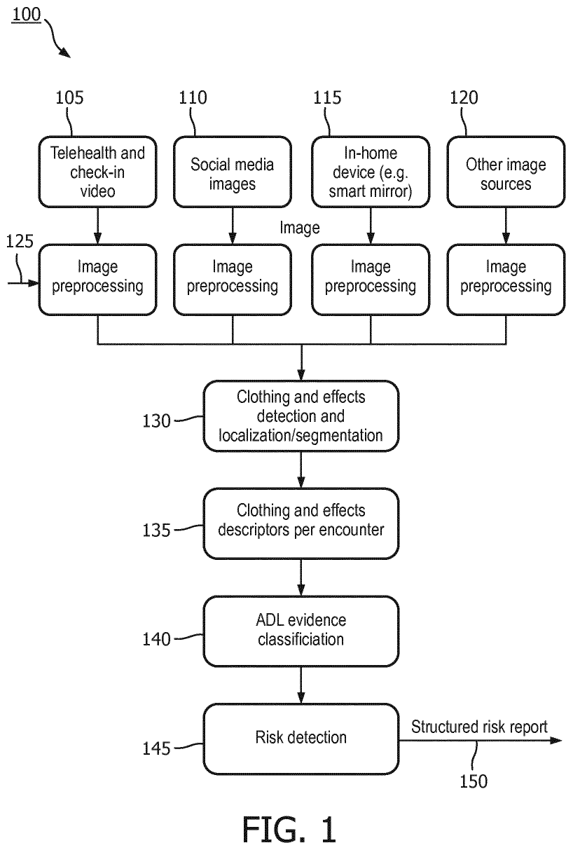

FIG. 1 illustrates a system overview 100 of different stages of monitoring, processing, and reporting deficiencies in ALDs in accordance with embodiments described herein.

A set of image sources 105-120 may produce still images and/or frames from still or video feeds with a greater than a specified probability (e.g. ninety percent) of being the subject at an appropriate time and place (i.e., when he/she would typically have completed dressing and personal hygiene ADLs), and with time and location metadata included.

The multiple image sources 105-120 may be used either individually or in combination to improve a quantity and variety of potential ADL deficiency evidence. Image sources 105-120 may provide images continuously (e.g., from a continuous video feed) or at regularly-spaced time intervals, although images may be timestamped, and greater frequency of images may improve risk assessments. Several different mechanisms may be used to input images or video for a machine vision system to analyze and make determinations re ADL deficiencies.

As illustrated in FIG. 1, image sources 105-120 may include telehealth and wellness check-in video 105. A variety of Philips® and third-party services and solutions may involve regular video contact with care providers. Still images may be captured from these videos.

Using the telehealth and check-in video 105. A subject could be instructed to check in with an imaging system once or twice per day. The imaging system could take a snapshot of different views of the subject or a short video of the subject. For a subject categorized as ADL-capable, such a procedure is viable, and there are other avenues to obtain images if the subject does not check in regularly.

Embodiments may include social media 110 sharing of images, either via general-purpose social media (e.g., Facebook®, Instagram®, or the like) or special-purpose social media may be targeted at subjects and their immediate social network.

On social media 110, if a subject posts hourly, daily, or weekly pictures of themselves, these images may be accessed by the system and analyzed in a manner described herein to monitor changes in ADL-indicative appearance.

In-home smart devices 115 are capable of capturing images and may be placed in appropriate locations in a subject's home. For example, smart devices such as a “smart mirror” may be placed in a bathroom or bedroom to take pictures or videos of the subject. These devices may also have purposes in addition to image capture, which may increase technology acceptance.

In-home devices 115 could include various cameras positioned throughout a subject's home. For example, there could be a camera in every room, or less expensively, a camera in the few rooms where a subject frequents most, such as their bedroom, kitchen, and bathroom. An image source could include an electronic personal assistant such as the Amazon Echo® or the like, to capture images or video of a subject.

Other image sources 120 could include a subject's smartphone, personal computer, or tablet, which can be configured to capture at least one picture or video of a user throughout a day, and over a course of days, weeks, months, and years.

After capturing images and video, a set of image preprocessing components with at least one customized preprocessing module 125 for each image source 105, 110, 115, and 120, may standardize and filter the images produced by the image sources 105-120 yielding uniform images, with those unsuitable for reasons of image quality or other concerns (e.g., privacy) removed.

Fulfilling a daily requirement of images of a subject may be through engagement with the subject or through scheduled surveillance. With the check-in method, a subject may be instructed to check in at a certain time of day or night, or on some other regular schedule, through a series of checks. When this is not feasible or successful, any of the social media images 110, in-home devices 115, or other devices 120 may be used.

Preprocessing modules 125 may include modules having some common functionality, including filtering images for quality, resizing images to one or more standard formats, cropping images so that the subject is centered, and filtering images which include persons other than the subject. The preprocessing modules 125 may have a purpose of standardizing images across the different image sources 105-120.

Unsuitable or undesirable images could include those that may cause personal embarrassment or be of privacy concerns to a user. These undesirable images may be removed or distorted to preserve the desired content. Face identification methods may be used to determine a subject's face from a visitor's face. Undesirable images may also include images where the subject is not present, such as when a device 115 or 120 obtains an image at a certain time and misses capturing the subject.

Preprocessing modules 125 may process telehealth and wellness check-in video sources 105 to select one or more “good” frames from a video, optimizing criteria such as image quality and the subject's positioning in the frame.

Similar processing may be performed on social media 110 images that may be less tightly time- and location-constrained than other sources (it is common to upload images later, sometimes much later, than when they are captured), and the preprocessing module 125 may attempt to detect time-shifted and location-shifted images by examination of image metadata or of image content.

In-home devices 115 such as smart mirrors have additional privacy concerns, such as capturing an image of the subject while undressed. Filtering may be applied by a preprocessing module 125 to detect and avoid these images.

A module or component as described herein may include any type of processor, computer, special purpose computer, general purpose computer, image processor, ASIC, computer chip, circuit, or controller configured to perform the steps or functions described therewith.

Embodiments may provide different options regarding where the image preprocessing module 125 is performed. Image preprocessing modules 125 may be located within devices 105-120 at a subject's home or residence. Devices 105-120 may use the preprocessing module 125 to perform the preprocessing or the devices 105-120 may transfer images to a computer system or server at the subject's home, and the computer system or server may store the image and conduct preprocessing thereon. Alternatively, images captured from devices 105-120 may be sent to a central server at a remote location where preprocessing modules 125 perform preprocessing. Images may be transmitted wirelessly, through the internet, or on computer readable media.

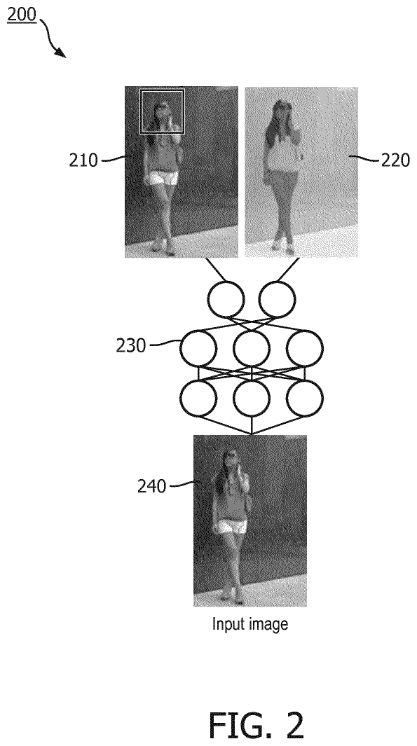

FIG. 2 illustrates a multi-task convolutional neural network (CNN) 200 configured to perform face-clothing detection and clothing segmentation in accordance with FIG. 1. After standardized images 240 are obtained by the preprocessing modules 125, clothing and effects segmentation/localization module 130 may include the CNN to separate clothing and personal effects via segmentation and/or localization. Within this analysis, a face may be simultaneously segmented when both detection and segmented are performed together. A deep CNN 230 inputs the image 240 and outputs pixel-wise labels 220 of clothing, hair, and accessories, and a bounding box 210 around a face.

A cropped face region out of face detection may be fed into a recognition module, which can be either based on handcrafted face feature matching (e.g., Eigenface@) or the deep convolutional neural networks. (e.g., DeepFace®). In this way, face identification may ensure that a correct subject is being monitored so that false negatives are not triggered by data acquired on family members or care givers. As noted, outputs of this component may include images with associated “masks” indicating which pixels of the image are clothing 220 and personal effects and/or bounding boxes 210 around regions of interest.

Attributes such as color, texture, materials, etc., may be extracted from the segmented clothing regions and compared with that of the reference clothing, which can be taken off days ago or provided by the end-user. Clothing change can be noted if the attribute differences of the captured and referred ones are larger than a tunable threshold. Certain changes (or lack thereof) may then be classified as ADL evidence, which is used with other evidence to calculate a risk score. In a case of clothing, if the CNN 200 detects no change over a period longer than 1 or 2 days, evidence may be logged. If no change is detected this may be used as evidence of declining capabilities.

In the clothing and effects segmentation/localization module 130, preprocessed images may be identified and classified into different groups for comparison with stored images. Images may be classified into clothing groups of the subject, facial and body images of the subject, embarrassing or unusable images of the subject, images that are not the subject, and images of blank space that do not include the subject. These groups may be further analyzed and divided into subgroups depending on characteristics of the group such as type of clothing, areas of a subject's anatomy, and so forth.

After implementing algorithms to detect clothing and personal effects, the localization and segmentation module 130 may identify an individual space as a preprocessing step for what areas of interest are in the images to get classified.

The localization and segmentation module 130 may yield a description of an image with particular regions marked out, of interest. Data may flow into the same classifiers that perform classification of what clothes someone is wearing, whether the clothes are dirty or disheveled, or whether their hair is messy. At a minimum, the localization and segmentation module 130 yields a presentation of the clothing someone is wearing. Localized regions of interest are identified.

Localized regions of interest are input to a clothing and effects descriptors per encounter module 135.

An ADL evidence classification component 140 may classify segmented images from the localization and segmentation module 130 and output scores (estimated probabilities) for the presence or absence of one or more categories of evidence of ADL deficiency, such as (a) dirty, wrinkly, or disheveled clothing in single images. The ADL evidence classification component 140 takes as input the segmented images from the module 130 and/or image features output by the previous component 135. Other categories may include (b) un-brushed, or messy hair, and (c) the same items of clothing worn on multiple days, in a sequence of images.

In ADL evidence classification, machine learning models may be applied to the sequences of images to classify them as containing or lacking multiple types of evidences of ADL deficiency. Several types of evidence are described herein, but embodiments are not limited thereto.

Dirty or disheveled clothing, hair, and personal effects may be classified using the deep CNN model 230, or a similar model, augmented with additional layers for attribute recognition. The structure of this model is similar to FIG. 2 above, but with only a single output.

During ADL evidence classification 140, change of clothing is classified using methods in which a classifier is used to match features including hue, saturation, value (HSV) color, 2D color histograms (e.g., LAB color space), superpixel geometric ratios, superpixel feature similarities, edges, textures, and contours. Repeated wearing of clothing may be identified when no change of clothing in a sequence of images spanning a specified time period.

The ADL evidence classification module 140 may translate the information into evidence for or against ADL, including raw scores of whether clothes are dirty or disheveled. The module 140 makes a temporal comparison which examines a similarity of different articles of clothing to estimate the probability that two or more time related clothing items are the same. ADL evidence scores may be produced.

After classification, a risk detection module 150 may detect when ADL evidence scores indicate the presence of ADL deficiency with increased risk of adverse events (e.g., when cumulative ADL deficiency evidence is above a specified threshold) and produces a structured risk report describing the ADL deficiency, and the resultant risk. A threshold may, for example, be three instances within one week, or other value or time period. Embodiments may create a structured report with elements including a summary of the amount and type of evidence of ADL deficiency, a description of the resulting risk, and annotated images from which ADL evidence was identified. The risk report may be delivered to formal or informal caregivers and actions may be taken commensurate therewith.

The risk detection module 145 may perform an algorithm that applies one of several risk models that predicts various kinds of risks from performance vectors of daily living, such as if someone is dressing themselves. The ADL scores contribute to the risk of several adverse events.

If a risk is determined, a home health care facility could be contacted that sends a worker to check on the subject being monitored. Also, information about the subject could be entered into a database to be catalogued with previously stored information. This information could be used in the future to determine a proper course of action.

The risk detection model may be rule based, including a weighted average of data, or a weighted logistic progression. The risk detection may include a simple score calculation. The risk detection model may include a clinical research editor. Surveys may include activity performance and subsequent events.

Embodiments include several novel and visible elements, including the use of captured images or video of a state of clothing and effects for ADL assessment, especially if without any observation or capture of the performance of ADLs directly. Embodiments are focused on assessment of dressing and personal hygiene ADLs and the generation of a structured risk report with annotated visual evidence.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A method of detecting decline in activities of daily living (ADLs) over time, the method comprising:
receiving a previously gathered, and stored plurality of image data of a subject over a period of time;
preprocessing the image data to obtain a plurality of standardized images;
segmenting out a feature from each of the image data, wherein the feature comprises an article of clothing or a personal effect;
providing the segmented features to a trained model to identify possible changes in the features over time, wherein possible changes in the segmented feature are identified by matching features over time, wherein the features comprise one or more of a hue, a saturation, a value color, a 2D color histogram, a superpixel geometric ratio, a superpixel feature similarity, an edge, a texture, and a contour;
classifying the possible changes as evidence; and
using the evidence to calculate a risk score.

2. The method of claim 1, wherein the image data is still image data.

3. The method of claim 1, wherein the image data is video image data.

4. The method of claim 1, wherein the feature comprises a bodily feature.

5. The method of claim 1, wherein the trained model is a convolutional neural network (CNN).

6. The method of claim 5, wherein the CNN detects the possible changes as no change over a threshold period as evidence of declining ADL capabilities.

7. The method of claim 1, wherein the risk score is reported to a health care management entity.

8. The method of claim 1, comprising:
detecting a lack of personal hygiene and repeated use of clothing based on the segmented features; and
determining that the lack of personal hygiene and repeated use of clothing are evidence of an ADL deficiency.

9. The method of claim 8, wherein the detecting includes capturing images of a same clothing item over at least three days.

10. A detection system, comprising:
a plurality of image sources to obtain a plurality of images of a subject at periodic intervals; at least one image preprocessing module configured to preprocess the plurality of images to obtain standardized images;
a segmentation component configured to apply techniques to the plurality of images to separate a feature of the plurality of images via segmentation, wherein the feature comprises an article of clothing or a personal effect;
a processor adapted to identify possible changes in the features over time by way of a trained model, wherein possible changes in the segmented feature are identified by matching features over time, wherein the features comprise one or more of a hue, a saturation, a value color, a 2D color histogram, a superpixel geometric ratio, a superpixel feature similarity, an edge, a textures, and a contours; and
an activity of daily living (ADL) evidence classification module configured to classify the possible changes as evidence and use the evidence to calculate a risk score for or against ADL deficiencies.

11. The detection system of claim 10, wherein the images are from still or video feeds.

12. The detection system of claim 10, wherein the image sources include one of telehealth and check-in video, social media, or in-home devices.

13. The detection system of claim 10, wherein the image sources provide images at scheduled time intervals.

14. The detection system of claim 10, wherein the detection system is configured to produce images with a greater than ninety percent probability, or other specified probability, of being the subject at an appropriate time and place

15. The detection system of claim 10, wherein outputs from the segmentation component include images with associated masks to indicate which pixels of the image are clothing and personal effects and/or bounding boxes around a region of interest.

16. The detection system of claim 10, wherein in segmentation component, preprocessed images are identified and classified into different groups for comparison with stored images.

17. The detection system of claim 10, wherein images are classified into clothing groups of the subject, facial and body images of the subject, embarrassing or unusable images of the subject, images that are not the subject, and images of blank space that do not include the subject.

18. The detection system of claim 10, wherein the ADL evidence classification module comprises a temporal comparison module which examines similarity of different articles of clothing to determine whether two or more time related clothing items are the same.

19. The detection system of claim 10, wherein the ADL evidence classification module is configured to produce raw scores of whether clothes are dirty or disheveled.

20. The detection system of claim 10, comprising a risk detection component configured to identify a risk whenever cumulative ADL deficiency evidence is above a specified threshold within a specified time period.

21. The detection system of claim 10, comprising a risk detection module configured to detect when ADL evidence indicates the presence of ADL deficiency with increased risk of adverse events.

22. The detection system of claim 10, comprising a risk detection module to produce a structured risk report when cumulative ADL deficiency evidence is above a specified threshold, the structured risk report describing the ADL deficiency and a resultant risk.

23. The detection system of claim 22, wherein the risk report is annotated with images of ADL evidence that was detected.
